(19) United States
(12) Patent Application Publication
(10) Pub. No.: US 2013/0237716 A1
(43) Pub. Date: Sep. 12, 2013

(54) Expression Modulator For Clock Gene

(71) Applicants: Yoko Gozu, Yokohama-shi (JP); Shinichiro Haze, Yokohama-shi (JP)

(72) Inventors: Yoko Gozu, Yokohama-shi (JP); Shinichiro Haze, Yokohama-shi (JP)

(73) Assignee: SHISEIDO COMPANY, LTD., Tokyo (JP)

(21) Appl. No.: 13/422,008

(22) Filed: Mar. 16, 2012

(30) Foreign Application Priority Data
Mar. 7, 2012 (JP) 050942/2012

(51) Int. Cl. C07C 69/78; C07D 303/04; C07C 229/60

(52) U.S. Cl. 549/545; 560/19; 560/103

(57) ABSTRACT

The present invention provides substances that can be used in a broad range of applications and effectively regulate clock gene expression.

Provided is a clock gene expression regulating agent comprising, as an active ingredient, one or more selected from the group consisting of caryophyllene oxide and compounds represented by the following structural formula:

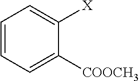

wherein X is CH₃, NH₂ or NHCH₃.

### TECHNICAL FIELD

The present invention relates to a clock gene expression regulating agent, and to a circadian rhythm modulator containing the clock gene expression regulating agent.

### BACKGROUND ART

Almost all organisms on the earth have an internal “biological clock” that oscillates automatically on a 24-hour cycle. The biological clock causes diurnal (circadian) fluctuations called “circadian rhythms”, and is thought to control diurnal fluctuations in various biological phenomena (activities) including not only the biological sleeping and waking cycles, but also body temperature, blood pressure, hormone secretion and metabolism as well as mental and physical activity, food intake and the like. In recent years, disturbances in circadian rhythms have been implicated in a variety of mental and physical symptoms and disorders, including sleep disorders, skin diseases and lifestyle-related diseases as well as depression and other neuropsychiatric disorders and the like.

As shown in FIG. 1, the biological clock is controlled by a rhythm-generating system that comprises genes called “clock genes”, and in mammals a feedback loop formed by transcriptional activation and suppression of genes coding for 4 core proteins called CLOCK, BMAL1, PERIOD and CRYPTOCHROME forms the core of the molecular clock mechanism. This feedback loop operates on a roughly 24-hour cycle, thereby creating circadian rhythms.

Clock genes directly control the expression rhythm of other genes as transcription factors, and indirectly control the diurnal expression of many more genes by regulating hormone secretion and the like, and it has been shown that when the expression rhythms of clock genes are disrupted in the body, the functions of bodily organs and the endocrine system are disrupted, thereby causing a variety of diseases including high blood pressure and lifestyle-related diseases (Non-patent Document 1). For example, abnormal expression of clock genes has been reported in the obese, and has also been associated with depression, cancer and the like. Clock genes are also known to regulate the circadian rhythms of various physiological functions of the skin, and it has been reported that in testing using normal human skin fibroblast cells, the type-I collagen gene is expressed in a circadian rhythm with an expression pattern similar to that of the clock gene Period 2 (Non-patent Document 2).

The circadian rhythm control center (central clock) is located in the suprachiasmatic nucleus of the hypothalamus, but it has been shown that clock genes are also expressed in the liver, kidneys, skin and other peripheral tissues, where they form circadian rhythms by a similar system. Although expression of clock genes in the periphery is regulated by signals from the superchiasmatic nucleus, signal stimulation factors such as glucocorticoids, catecholamine, angiotensin II and the like have also been shown to produce physiological rhythms by directly controlling clock gene expression in peripheral tissues and cells. In recent years, glucocorticoids, forskolin, serum and other stimulation factors have been added to skin fibroblasts and other cultured cells to synchronize expression rhythms and induce circadian rhythms of clock gene expression in vitro, and this expression has been used as a marker to evaluate circadian rhythms (Non-patent Documents 3 to 5).

In similar evaluation systems using human skin fibroblasts, it has been reported that hinoki cypress extract, chlorella extract and other natural medicines and juniper oil, lavender oil and other essential oils can regulate expression of Bmal clock genes, and that arnica extract, Nuphar japonica and other natural medicines and juniper oil, cedar oil and other essential oils can regulate expression of Period clock genes (Patent Documents 1 and 2).

As discussed above, various behavioral rhythms and circadian rhythms of physiological functions in the body can be regulated by regulating the expression of the clock genes that control them, and there is strong demand for the development of new drugs that can be used in a broad range of applications and are capable of effectively regulating clock gene expression.

### [Patent Document 1]

International Publication No. WO 2011/122040

### [Patent Document 2]

International Publication No. WO 2011/122041

### [Non-patent Document 1]

Hastings M., O'Neill J. S., and Maywood E. S., (2007) Circadian clocks: regulators of endocrine and metabolic rhythms. Journal of Endocrinology, 195: 187-198

### [Non-patent Document 2]

Izumi Katsuyo et al, Gaijitsu rizumu wo motsu hifuseiriidennshi no tansaku (Search for Skin Physiological Genes with Circadian Rhythm), The Molecular Biology Society of Japan, 32nd Annual Meeting, Abstract 2P-0009

### [Non-patent Document 3]

Okamura H., (2004) Clock genes in cell clocks: Roles, Actions, and Mysteries. Journal of Biological Rhythms, 19(5): 388-399

### [Non-patent Document 4]

Balsalobre A., Damiola F., and Schibler U., (1998) A serum shock induces circadian gene expression in mammalian tissue culture cells. Cell, 93: 929-937

### [Non-patent Document 5]

Yagita K., Tamanini F., van der Horst G., and Okamura H., (2001) Molecular mechanisms of the biological clock in cultured fibroblasts. Science, 292: 278-281

### DISCLOSURE OF THE INVENTION

In light of the circumstances described above, it is an object of the present invention to provide a new drug capable of regulating clock gene expression.

The inventors achieved the present invention by discovering that in cultured cells, caryophyllene oxide and methyl benzoate derivatives can promote expression of Bmal, Period and other clock genes.

The clock gene expression regulating agent of the present invention comprises, as an active ingredient, one or more selected from the group consisting of caryophyllene oxide and compounds represented by the following structural formula:

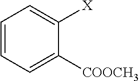

wherein X is CH₃, NH₂ or NHCH₃.

Previously the fact that these specific compounds can regulate expression of clock genes was entirely unknown.

Although this is not a limitation, the clock gene expression regulating agent of the present invention regulates expression of the Bmal genes (Bmal1, Bmal2), Period genes (Period1, Period2, Period3), Clock gene, Cryptochrome gene, albumin site D-binding protein (Dbp) gene, E4BP4 gene, Npas2 gene and Rev-erb gene, and of these, it regulates expression of the Bmal, Period, Clock and/or Cryptochrome genes, which are core genes of biological clocks, and especially the Bmal and Period genes.

In the present invention, gene expression regulation includes not only promotion of gene expression but also regulation of gene expression rhythms (phase or period).

The circadian rhythm modulator of the present invention contains the aforementioned clock gene expression regulating agent. As discussed above, clock genes directly or indirectly control the circadian expression of various genes associated with organ function and the endocrine system in the body, and can modulate various behavioral rhythms and the rhythms of physiological function in the body by regulating the expression of clock genes that control these functions, such as Bmal and Period genes, which are core genes of biological clocks.

The clock gene expression regulating agent of the present invention can be used in a variety of embodiments including drugs, quasi-drugs, cosmetics, foods, general merchandise, clothing and the like, and can improve various mental and physical symptoms and diseases caused by disruption of circadian rhythms.

### BRIEF DESCRIPTION OF THE DRAWINGS

FIG. 1 is a schematic diagram showing the core loop part of a circadian rhythm generating system by clock genes.

FIG. 2 is a graph showing induction of circadian rhythms of clock gene expression with cortisol in cultured human skin fibroblast cells.

FIG. 3A is a graph showing expression regulation of the clock gene Bmal1 by test substances in cultured human skin fibroblast cells.

FIG. 3B is a graph showing expression regulation of the clock gene Per1 by test substances in cultured human skin fibroblast cells.

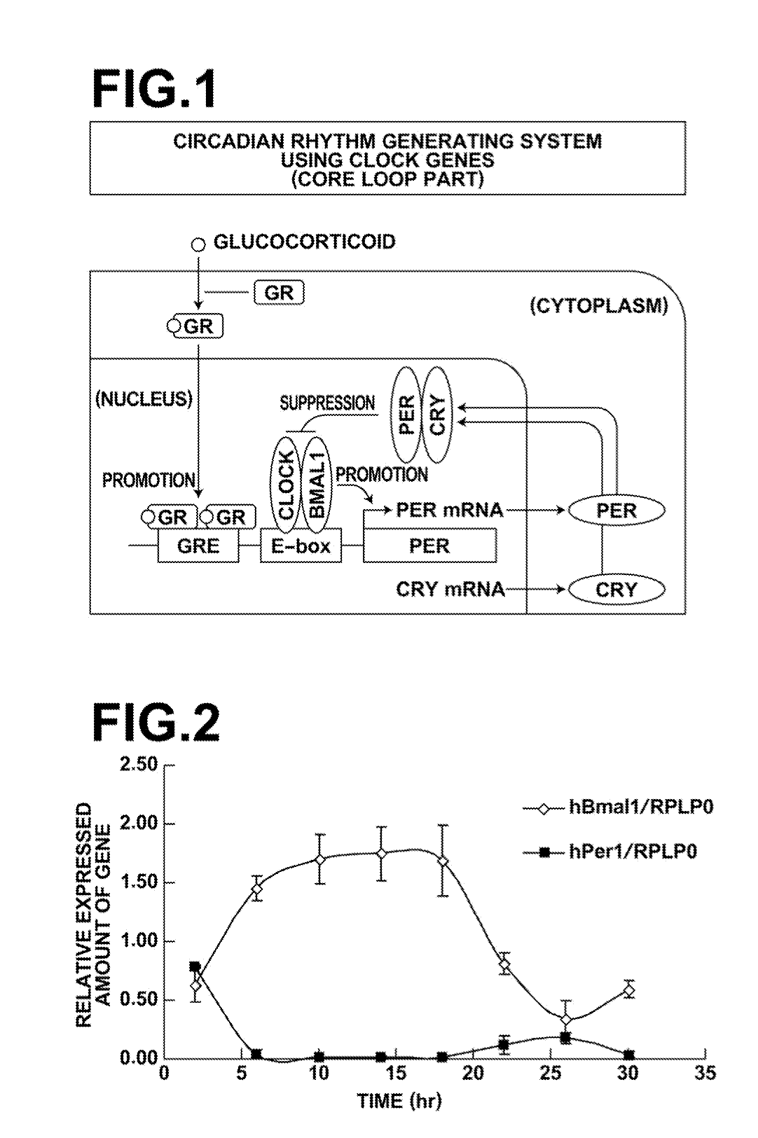

### BEST MODE FOR CARRYING OUT THE INVENTION

The clock gene expression regulating agent of the present invention comprises caryophyllene oxide or one of the aforementioned methyl benzoate derivatives as an active ingredient. These compounds are all known compounds, and are explained briefly below.

Caryophyllene oxide ((1R,4R,6R,10S)-4,12,12-trimethyl-9-methylene-5-oxatricyclo[8.2.0.04.6]dodecane) is a colorless liquid or colorless crystal with a molecular weight of 220.35, and is obtained by epoxylation of caryophyllene or isocaryophyllene with peracetic acid in the presence of sodium acetate at 0 to 5°. In nature, it is found in copaiba oil, clove oil, lavender oil and the like.

The methyl benzoate derivative methyl 2-methylbenzoate used in the present invention is a colorless liquid with a molecular weight of 150.18. Methyl 2-aminobenzoate is a liquid with a molecular weight of 151.17, and can be obtained by adding 80 parts of anthranilic acid to 120 parts of methanol, gradually adding 100 parts of concentrated sulfuric acid at 40° or below, completing the reaction at 75° to 83°, collecting the methanol, adding this to a solution of 110 parts of sodium carbonate and 240 parts of water to neutralize it, and then water washing and purifying by distillation. Another method is by synthesis from o-nitrotoluene. In nature, it is found in neroli, ylang-ylang flower oil and the like. Methyl 2-methylaminobenzoate is a colorless liquid with a molecular weight of 165.2, and is found in nature in orange peel, mandarin oil and the like.

The clock gene expression regulating agent of the present invention contains 1 or 2 or more of the aforementioned compounds. In the present invention, these compounds may be naturally derived, or may be synthetic compounds.

The clock gene expression regulating agent of the present invention may be used alone, or may be used in combination with other drugs having a clock gene expression regulating effect.

Moreover, the clock gene expression regulating agent and circadian rhythm modulator of the present invention may be used alone, or may be included in a variety of objects, and any components may be included in addition to the aforementioned essential component according to the type of the object.

For example, if the object is a topical skin preparation, any component commonly included in topical skin preparations can be included together with the aforementioned clock gene expression regulating agent according to the dosage form (for example, liquid, powder, granules, aerosol, solid, gel, patch, suppository or the like) or product type (for example, cosmetic, drug, quasi-drug or the like). The concept of a topical skin preparation encompasses all compositions that are applied to the skin (including scalp, hair and nails), such as base cosmetics, make-up, hair cosmetics, skin and hair washing products and other cosmetics as well as ointments, patches, suppositories, toothpastes and various other drugs and quasi-drugs and the like. The formulation is not particularly limited, and any form is possible including aqueous solutions, solubilized forms, emulsions, oils, gels, pastes, ointments, aerosols, water-oil 2-phase systems and water-oil-powder 3-phase systems for example. When the topical skin preparation is a cosmetic, it may be in the form of a perfume, eau de toilet, eau de cologne, cream, emulsion, skin lotion, foundation, face powder, lipstick, soap, shampoo or conditioner, body shampoo, body rinse, body powder, bath salts or the like.

Moreover, the clock gene expression regulating agent of the present invention can be included in an air freshener, deodorant, scented candle, incense, stationery product, pocketbook, bag, shoes or any other general merchandise, or in underwear, clothing, hats, stockings, socks or any other garments, or in powders, granules, tablets, capsules and various other kinds of supplements (nutritional supplements), or in confectionery, drinks and various other food products, or it can be used in an inhaled product such as an inhalant drug or air freshener.

Examples of the mode of use of the clock gene expression regulating agent of the present invention were given above, but these examples are not limiting, and any other mode of use can be adopted as long as it provides the effects of the present invention. In addition to the clock gene expression regulating agent of the present invention, other drugs having circadian rhythm modulating effects can be compounded according to the specific embodiment to the extent that they do not detract from the effects of the present invention.

The compositional amount of the clock gene expression regulating agent of the present invention in the object is not particularly limited, and can be selected appropriately according to the type and form of the compound used, the object and the like, but is for example 0.00001 mass % to 100 mass % or preferably 0.0001 mass % to 50 mass % or more preferably 0.0001 mass % to 20 mass % of the total mass of the object.

The specific application of the object in which the clock gene expression regulating agent or circadian rhythm modulator of the present invention is included is not particularly limited as long as it relates to the modulation of circadian rhythms. For example, it can be applied to the prevention, improvement, treatment or the like of jet lag syndrome, shift work syndrome, delayed sleep phase syndrome, non-24-hour sleep-wake disorder, depression with circadian rhythm sleep disorder and the like, as well as insomnia, poor physical condition, attention deficit, apathy, rough skin and various other symptoms that are associated with circadian rhythm disorder.

### EXAMPLES

The present invention is explained in detail below using examples, but the present invention is not limited to these examples. Skin fibroblast cells, epithelial cells, endothelial cells, pigment cells, fat cells, nerve cells and various other cells can be used as cultured cells, but in these examples evaluations were performed using human skin fibroblast cells. Because the clock gene core system is common to all species of organism and all types of cells, it is thought that evaluation results from human skin fibroblast cells are applicable to other species of organism and other types of cells. Known clock genes include Bmal, Period, Clock, Cryptochrome, albumin site D-binding protein (Dbp), E4BP4, Npas2 and Rev-erb genes and the like, but in the examples expression of Bmal and Period genes, which are involved in the core system, was measured. Bmal1 and Bmal2 are known as Bmal genes and Per1, Per2, Per3 as Period genes, but it is thought that members of the same gene group exhibit similar behaviors, and Bmal1 and Per1 were used as representative examples. In the examples below, human Bmal1 (hBmal1) and human Per1 (hPer1) expression was measured. The test substances used below were purchased as commercial products.

### Induction of Circadian Rhythm of Clock Gene Expression Using Cultured Human Skin Fibroblast Cells

It was confirmed that a circadian rhythm of clock gene expression can be induced in a system using cultured human skin fibroblast cells.

As the cultured human fibroblast cells, fibroblast cells from normal human skin were purchased (Cell Application, Inc.) and used in the experiments. They were seeded on DMEM medium with 10% EBS, 20 μM HEPES, Glutamax and an antibacterial agent added thereto, and cultured at 37° C. in 5% CO₂. On the 6th day of culture, 50 ng/ml of cortisol was added, and after 2 hours of treatment the cells were sampled over time. RNA was extracted from the cells with a commercial RNA extraction kit, and the expressed amounts of the hBmal1 and hPer1 genes were measured by RT-PCR using a commercial PCR primer (Perfect Real Time Primer, Takara Bio). Similarly, the expressed amount of the housekeeping gene RPLP0 was assayed and used as an internal standard to calculate the expressed amounts of the target genes relative to the expressed amount of RPLP0.

In the body, cortisol and other glucocorticoids are involved in regulating the biological clock in peripheral tissue and the like, and it is thought that blood concentrations of cortisol rise at the time of awakening in the morning, resetting the biological clock. In cultured cells, individual cells ordinarily keep a variety of different times, but stimulation with a signal stimulation factor such as cortisol serves to synchronize the clock gene expression rhythms, inducing a circadian rhythm.

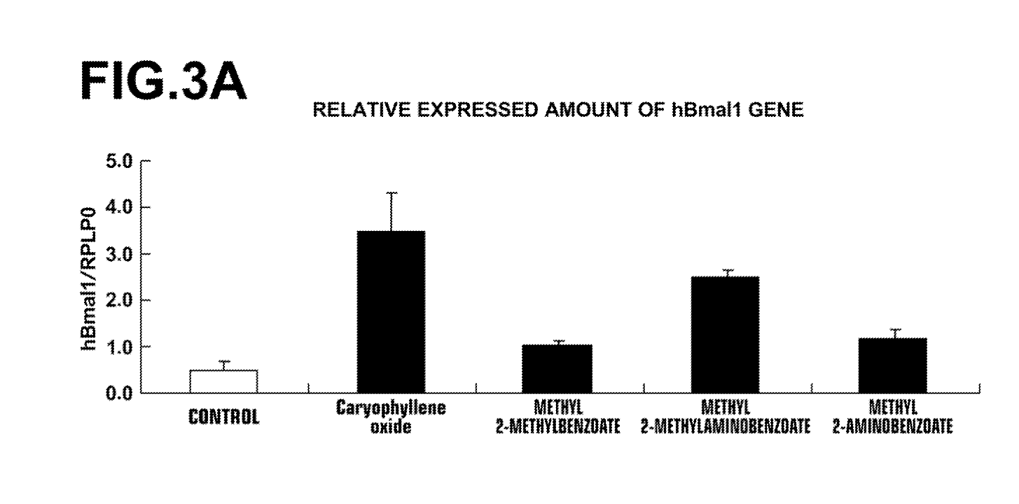

The results of hBmal1 and hPer1 measurement are shown in FIG. 2. It was confirmed that they were both expressed in a circadian rhythm with a roughly 24-hour cycle as a result of cortisol stimulation

### Evaluation of Clock Gene Expression Regulation Effects

The results above confirm that in an evaluation system using cultured human skin fibroblast cells, clock genes acquire a circadian rhythm when stimulated with cortisol. Because it is desirable to evaluate during a time period when the expressed amount is high, the clock gene expression regulation effects of the test substances were evaluated based on the expressed amount of the clock gene 16 hours after stimulation in the case of hBmal1 and 2 hours after stimulation in the case of hPer1.

Fibroblasts cells from normal human skin (Cell Application, Inc.) were seeded by methods similar to those used above, each chemical was added on the 6th day of culture to a concentration of 50 ppm, and the cells were sampled after 2 hours and after 16 hours. The same amount of ethanol was added instead of the test substance as a control. RNA was extracted from the cells with a commercial RNA extraction kit, and the expressed amounts of the hBmal1 and hPer1 genes were measured by RT-PCR using a commercial PCR primer (Perfect Real Time Primer, Takara Bio). Similarly, the expressed amount of the housekeeping gene RPLP0 was assayed and used as an internal standard to calculate the expressed amounts of the target genes relative to the expressed amount of RPLP0. Dunnett's multiple comparison test was performed on the obtained values, and an effect was recognized if there was a significant difference with a one-sided 5% risk factor in comparison with the control.

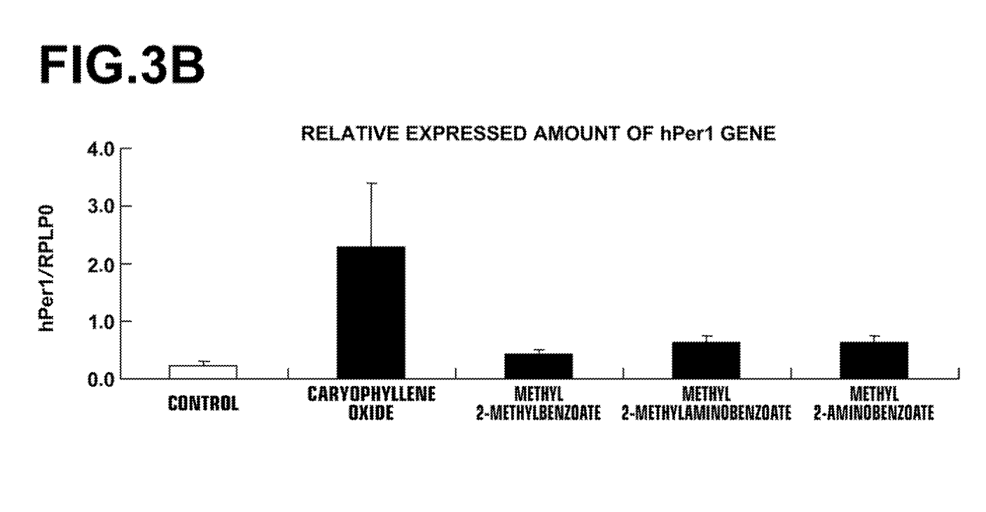

FIG. 3A shows the relative expressed amount of the hBmal1 gene 16 hours after addition of the test substance, and FIG. 3B shows the relative expressed amount of the hPer1 gene 2 hours after addition of the test substance.

Table 1 below shows the relative expressed amounts of the hBmal1 gene after 16 hours and the hPer1 gene after 2 hours.

TABLE 1

| | Relative expressed | Relative expressed |
|---|---|---|
| | amount of hBmall | amount of hPer1 |
| Test substance | (16 hours) | (2 hours) |
| Control | 0.48 | 0.24 |
| Caryophyllene oxide | 3.50* | 2.29* |
| Methyl 2-methylbenzoate | 1.01* | 0.44* |
| Methyl 2- | 2.52* | 0.64* |
| methylaminobenzoate | | |
| Methyl 2-aminobenzoate | 1.19* | 0.64* |

*p < 0.05

Caryophyllene oxide, methyl 2-methylbenzoate, methyl 2-methylaminobenzoate and methyl aminobenzoate (methyl anthranilate) significantly increased the expressed amounts of both the hBmal1 gene and hPer1 gene in comparison with the control, showing that these compounds are capable of regulating clock gene expression.

### COMPOSITIONAL EXAMPLES

Compositional examples of the clock gene expression regulating agent of the present invention are given below, but embodiments of the present invention are not limited to the following. Compositional amounts are all expressed as mass % relative to the total product.

### [Fragrance]

| | |
|---|---|
| (1) Alcohol | 75.0 |
| (2) Purified water | remainder |
| (3) Dipropylene glycol | 5.0 |
| (4) Caryophyllene oxide (clock gene expression | 10.0 |
| regulating agent of invention) | |
| (5) Antioxidant | 8.0 |
| (6) Dye | as needed |
| (7) UV absorbent | as needed |

### [Room Freshener]

| | |
|---|---|
| (1) Alcohol | 80.0 |
| (2) Purified water | remainder |
| (3) Antioxidant | 5.0 |
| (4) Methyl 2-methylbenzoate (clock gene expression | 3.0 |
| regulating agent of invention) | |
| (5) 3-methyl-3-methoxybutanol | 5.0 |
| (6) Dibenzylidene sorbitol | 5.0 |

### [Incense]

| | |
|---|---|
| (1) Makko powder | 75.5 |
| (2) Sodium benzoate | 15.5 |
| (3) Methyl 2-methylaminobenzoate (clock gene expression | 5.0 |
| regulating agent of invention) | |
| (4) Eucalyptus oil | 1.0 |
| (5) Purified water | remainder |

### [Bath Salts]

| | |
|---|---|
| (1) Sodium sulfate | 45.0 |
| (2) Sodium bicarbonate | 45.0 |
| (3) Lavender oil | 9.0 |
| (4) Methyl 2-aminobenzoate (clock gene expression | 1.0 |
| regulating agent of invention) | |

### [Massage Gel]

| | |
|---|---|
| (1) Erythritol | 2.0 |
| (2) Caffeine | 5.0 |
| (3) Phellodendron amurense bark extract | 3.0 |
| (4) Glycerin | 50.0 |
| (5) Carboxyvinyl polymer | 0.4 |
| (6) Polyethylene glycol 400 | 30.0 |
| (7) Trisodium edetate | 0.1 |
| (8) Polyoxylene (10) methylpolysiloxane copolymer | 2.0 |
| (9) Squalane | 1.0 |
| (10) Sodium hydroxide | 0.15 |
| (11) Caryophyllene oxide (clock gene expression | 0.5 |
| regulating agent of invention) | |
| (12) Methyl 2-methylbenzoate (clock gene expression | 0.5 |
| regulating agent of invention) | |

### [Massage Cream]

| | |
|---|---|
| (1) Solid paraffin | 5.0 |
| (2) Beeswax | 10.0 |
| (3) Vaseline | 15.0 |
| (4) Fluid paraffin | 41.0 |
| (5) 1,3-butylene glycol | 4.0 |
| (6) Glycerin monostearate | 2.0 |
| (7) POE (20) sorbitan monolaurate ester | 2.0 |
| (8) Borax | 0.2 |
| (9) Caffeine | 2.0 |
| (10) Preservative | as needed |
| (11) Antioxidant | as needed |
| (12) Caryophyllene oxide (clock gene expression | 0.5 |
| regulating agent of invention) | |
| (13) Methyl 2-methylaminobenzoate (clock gene expression | 0.5 |
| regulating agent of invention) | |
| (14) Purified water | remainder |

### [Aromatic Fiber]

Microcapsules containing the clock gene expression regulating agent of the present invention (particle diameter no greater than 50 μm, percentage of compound in microcapsule 50 wt %) were added to a cuproammonium cellulose solution (cellulose concentration 10 wt %, ammonium concentration 7 wt %, copper concentration 3.6 wt %) in the range of 0.1 wt % to 20 wt % of the cellulose weight, mixed, and spun by a conventional wet spinning method, and aromatic fiber was obtained by a refining step and a drying step.

### [Granules]

| | |
|---|---|
| (1) Sucralose | 0.1 |
| (2) Methyl 2-aminobenzoate (clock gene expression | 0.1 |
| regulating agent of invention) | |
| (3) Methyl 2-methylbenzoate (clock gene expression | 0.1 |
| regulating agent of invention) | |
| (3) Flavoring | 5.0 |
| (4) Excipient (Ceolus) | 10.0 |
| (5) Maltitol | remainder |

### [Tablets (Chewable Type)]

| | |
|---|---|
| (1) Inositol | 11.0 |
| (2) Maltitol | 21.0 |
| (3) Sucrose | 0.5 |
| (4) Salmon roe extract (DNA Na) | 0.1 |
| (5) Enzyme extract | 0.1 |
| (6) Methyl 2-aminobenzoate (clock gene expression | 0.1 |
| regulating agent of invention) | |
| (7) Methyl 2-methylaminobenzoate (clock gene expression | 0.1 |
| regulating agent of invention) | |
| (7) Flavoring | 5.0 |
| (8) Excipient | remainder |

### [Tablets]

| | |
|---|---|
| (1) Lubricant (sucrose fatty acid ester, etc.) | 1.0 |
| (2) Gum arabic aqueous solution (5%) | 2.0 |
| (3) Acidulant | 1.0 |
| (4) Colorant | as needed |
| (5) Methyl 2-methylbenzoate (clock gene expression | 0.1 |
| regulating agent of invention) | |
| (5) Methyl 2-methylaminobenzoate (clock gene expression | 0.1 |
| regulating agent of invention) | |
| (6) Sugars (powdered sugar, sorbitol, etc.) | remainder |

### [Candy]

| | |
|---|---|
| (1) Sugar | 50.0 |
| (2) Starch syrup | 47.95 |
| (3) Organic acids | 2.0 |
| (4) Methyl 2-aminobenzoate (clock gene expression | 0.05 |
| regulating agent of invention) | |

### [Gum]

| | |
|---|---|
| (1) Sugar | 43.0 |
| (2) Gum base | 30.95 |
| (3) Glucose | 10.0 |
| (4) Starch syrup | 16.0 |
| (5) Methyl 2-methylbenzoate (clock gene expression | 0.05 |
| regulating agent of invention) | |

When the products of these compositional examples were usage tested in a typical usage mode for each type of product, they were able to regulate clock gene expression and modulate circadian rhythms.

1. A clock gene expression regulating agent comprising, as an active ingredient, one or more selected from the group consisting of caryophyllene oxide and compounds represented by the following structural formula: 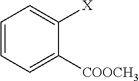
wherein X is CH₃, NH₂ or NHCH₃.

2. The clock gene expression regulating agent according to claim 1, wherein the clock gene is Bmal or Period.

3. A circadian rhythm modulator, containing the clock gene expression regulating agent according to claim 1.

4. A circadian rhythm modulator, containing the clock gene expression regulating agent according to claim 2.
